(19) United States
(12) Patent Application Publication
(10) Pub. No.: US 2004/0193262 A1
(43) Pub. Date: Sep. 30, 2004

(54) Implants for treating ocular hypertension, methods of use and methods of fabrication

(76) Inventor: John H. Shadduck, Tiburon, CA (US)

Correspondence Address: John H. Shadduck, 1490 Vistazo West, Tiburon, CA, 94920, US

(21) Appl. No.: 10/770,436

(22) Filed: Feb. 3, 2004

Related U.S. Application Data
(63) Continuation-in-part of application No. 10/759,797, filed on Jan. 17, 2004.
(60) Provisional application No. 60/459,196, filed on Mar. 29, 2003.
(60) Provisional application No. 60/469,783, filed on May 12, 2003.
(60) Provisional application No. 60/459,784, filed on Apr. 1, 2003.

(51) Int. Cl. A61F 2/14

(52) U.S. Cl. 623/4.1; 604/8; 623/23.7

(57) ABSTRACT

A stent for treating ocular hypertension by providing means for enhancing outflows of aqueous humor from the anterior chamber. An exemplary stent is fabricated of a shape memory polymer (SMP) that can withstand very large reversible inelastic strains for storing energy in a temporary reduced cross-sectional shape. In one embodiment, the stent in a temporary shape is introduced into a targeted tissue volume in and about the eye's aqueous outflow pathways. Following minimally invasive implantation of the stent, body temperature or another stimulus causes the stent to move from its temporary shape to its memory shape thereby releasing stored energy to retract the tissue to open flow pathways or increase tissue permeability. In another embodiment, the SMP stent body has interior flow passageways to provide addition fluid outflow means. In several embodiments, the stent can be of a shape memory alloy material.

### CROSS-REFERENCE TO RELATED APPLICATIONS

This application claims benefit of the following Provisional U.S. Patent Applications: Ser. No. 60/459,196 filed Mar. 29, 2003 titled Implantable Stent and Methods for Treating Glaucoma; Ser. No. 60/469,783 filed May 12, 2003 titled Intraocular Stents for Treating Glaucoma, Methods of Use and Methods of Fabrication; and Ser. No. 60/459,784 filed Apr. 1, 2003 titled Implants and Methods for Treating Glaucoma. This application also is a Continuation-in Part of U.S. patent application Ser. No. 10/759,797 filed Jan. 17, 2004 titled Implants for Treating Ocular Hypertension, Methods of Use and Methods of Fabrication. This application is related to the following Provisional U.S. Patent Applications: Ser. No. 60/425,969 filed Nov. 13, 2002 titled Implants and Methods for Treating Glaucoma; Ser. No. 60/424,869 filed Nov. 7, 2002 titled Implants and Methods for Treating Glaucoma, and Ser. No. 60/422,646 filed Oct. 31, 2002 titled Electrosurgical System Utilizing Thermoscissile Polymeric Compositions. All of the above Non-Provisional and Provisional U.S. Patent Applications are specifically incorporated herein in their entirety by this reference.

### BACKGROUND OF THE INVENTION

1. Field of the Invention

A stent or implant body for reducing ocular hypertension, and more in particular, a shape memory stent and method for enhancing outflows of aqueous humor from the anterior chamber by retracting tissue and modifying tissue properties within aqueous outflow pathways to thereby provide a less compacted tissue geometry and increased fluid permeability about the aqueous outflow routes.

2. Description of the Related Art

Glaucoma is the second leading cause of legal blindness in the United States with approximately 80,000 people in the United States being legally blind as a result of glaucoma. Large numbers of people suffer lesser visual impairment, with the American Academy of Ophthalmology reporting that approximately 2 million persons in the United States have primary open angle glaucoma (POAG, a common form of glaucoma). As many as seven million office visits are made annually in the U.S. for glaucoma diagnosis and treatments.

Glaucomas are a group of eye diseases that are characterized by elevated intraocular pressure (IOP) that causes a pathological change in nerve fiber layers of the retina resulting in losses in the field of vision. In a healthy eye, the ciliary body produces aqueous humor which circulates from the posterior chamber to the anterior chamber. The aqueous flows outwardly and exits the anterior chamber through the trabecular meshwork and the Schlemm's canal, which is located about the periphery of the anterior chamber, as well as through the region of the uveoscleral plane. If the aqueous outflow paths are obstructed or exhibit reduces fluid permeability, an excess of aqueous humor will be present in the anterior chamber and can lead to elevated intraocular pressure (IOP). The increased IOP associated with decreased aqueous outflows is a form of glaucoma that can lead to blindness.

Normal intraocular pressure is considered to be less than about 21-22 mm. Hg. However, as many as one in six patients with glaucoma have pressure below 21-22 mm. Hg and yet still have progressive eye damage. Further, in any single diagnostic test, as many as one-half of glaucoma patients will exhibit normal IOP levels but will actually average to have IOPs that are greater than 21-22 mm. Hg following repeated screening. Various surgical procedures and implant devices have been developed for treating glaucoma by increasing the rate of outflows of aqueous humor from the anterior chamber. None of the commercially available shunts and outflow devices has been widely accepted, and most require invasive surgery.

What is needed is a reliable implant device and method for treating ocular hypertension. In particular, what is needed is an implantable device that can be introduced into the eye in a simplified, minimally invasive procedure. Of particular importance, it would be desirable to have an implant that is inexpensive and that can be implanted by health care personnel worldwide that does not require highly specialized surgical skills. A large number of glaucoma patients worldwide do not have access to IOP-lowering drugs or expensive glaucoma surgeries.

### SUMMARY OF THE INVENTION

In general, the stents corresponding to the invention are fabricated of shape memory polymer materials that treating ocular hypertension by modifying functional properties of targeted soft tissues in the region of aqueous humor outflow pathways. An exemplary stent is at least partly fabricated of a shape memory polymer (SMP) that can withstand very large reversible inelastic strains for storing energy in a temporary reduced cross-sectional shape. The method includes introducing at least one stent into the trabecular meshwork, uveoscleral plane or subconjunctival plane to retract, dissect and modify the targeted tissue volume. Following minimally invasive implantation of the stent, body temperature or another stimulus causes the stent to move from its temporary shape to its memory shape thereby releasing stored energy to retract and dissect the targeted tissue to open flow pathways or increase tissue permeability. In one exemplary embodiment, the SMP stent body is fabricated with flow passageways extending therethrough to provide addition fluid outflow means.

The stents corresponding to the invention advantageously can be introduced with a needle-like injector to thereafter move from a reduced cross-sectional shape to an expanded shape to modify tissue properties such as fluid permeability to enhance aqueous outflows.

The stents corresponding to the invention can retract tissue in the trabecular meshwork and plates overlying Schlemm's canal to enhance outflows therethrough.

The stents corresponding to the invention can dissect tissue in the subconjunctival plane to enhance outflows therethrough.

The stents corresponding to the invention advantageously can provide an outflow pathway to the lymphatic network in the subconjunctival plane.

The stents corresponding to the invention are optionally biodegradable to allow for repeat therapies.

The stents can carry polymer surface layers that biodegrade to release pharmacological agents.

The present invention will now be described herein with respect to the foregoing preferred embodiments including various examples. These embodiments are designed to illustrate the invention, and do not limit the invention as defmed by the claims.

### BRIEF DESCRIPTION OF THE DRAWINGS

FIG. 1 is a sectional perspective view of a patient's eye showing the targeted aqueous outflow tissues, the lymphatic network of the eye, and exemplary vectors for introduction of the “retracting” stents of the invention.

FIG. 2 is a sectional image of a human eye illustrating the tissue volumes targeted for retraction about the aqueous outflow pathways.

FIG. 3A is a perspective view of a stent corresponding to the invention, the stent fabricated of a shape memory polymer (SMP) in a helical form in a temporary non-extended shape, the stent adapted for in situ release of stored energy to retract tissue.

FIG. 3B is a perspective view of the stent of FIG. 3A in its memory extended shape.

FIG. 4 is a cut-away view of the eye and the stent of FIGS. 3A and 3B moving to its memory shape from its temporary shape to retract and modify tissue in the meshwork region.

FIG. 5A is a perspective view of an alternative stent of a SMP in a spiked form in a temporary non-extended shape.

FIG. 5B is a perspective view of the stent of FIG. 5A in its memory extended shape.

FIG. 6 is a cut-away view of the eye and the stent of FIGS. 5A and 5B in its memory shape after in situ release of stored energy to cause the spike portions to retract and modify tissue and to provide flow pathways.

FIG. 7A is a perspective view of an alternative stent of a SMP in an undulating form in a temporary non-extended shape.

FIG. 7B is a perspective view of the stent of FIG. 7A in its memory extended shape.

FIG. 8 is a sectional view of a portion of the eye with the stent of FIGS. 7A and 7B in its memory shape after in situ release of stored energy to retract and modify meshwork tissue.

FIG. 9A is a perspective view of an alternative stent system consisting of a plurality of SMP bodies in temporary non-extended configurations.

FIG. 9B is a perspective view of the SMP stent bodies of FIG. 9A in memory extended configurations.

FIG. 9C is a sectional view of a portion of an eye with the distributed stents of FIGS. 9A and 9B in memory shapes after in situ release of stored energy to retract and dissect the uveoscieral plane to modify tissue and provide enhanced permeability.

FIG. 9D is a sectional view of a portion of an eye with an alternative SMP stent for retracting and dissecting the uveoscieral plane to enhance fluid permeability.

FIG. 10A is a sectional view of an alternative SMP stent just after deployment in targeted tissue beginning to expand from its temporary non-extended shape.

FIG. 10B is a view of the SMP stent as in FIG. 10A after expansion to its memory extended shape to retract and modify the permeability of tissue.

FIG. 11 is a perspective view of an alternative stent of a shape memory alloy (SMA) body in a cut-away view of the eye in its memory extended shape.

FIG. 12 is a perspective view of the SMA stent of FIG. 12 showing its needle introducer means for constraining the SMA body.

FIG. 13 illustrates an alternative stent body with a first end portion of the stent fabricated of an expandable SMP portion for retracting tissue and modifying tissue with a second end portion fabricated of a non-SMP extension portion for carrying fluid outflows away from a modified tissue site.

FIG. 14 illustrates an alternative stent body similar to that of FIG. 13 with the medial portion fabricated of an expandable shape memory polymer.

FIG. 15 is a cut-away view of a portion of an eye with a stent similar to that of FIGS. 13 and 14 in a memory shape after in situ release of stored energy to retract and dissect the uveoscleral plane at a first end and the subconjunctival plane at the second end.

FIG. 16 illustrates a SMP stent body for treating angle-closure glaucoma by expanding the angle while at the same time allowing for fluid flows therethrough.

### DESCRIPTION OF PREFERRED EMBODIMENTS OF THE INVENTION

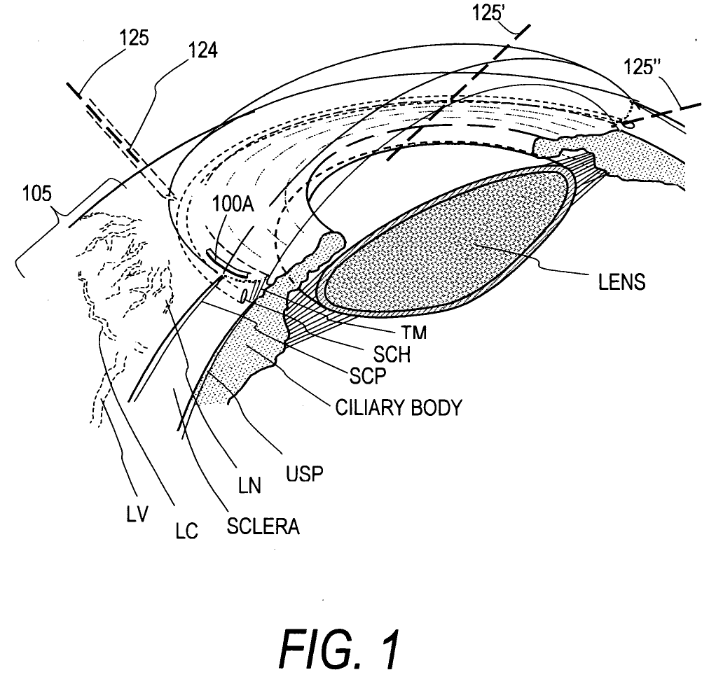

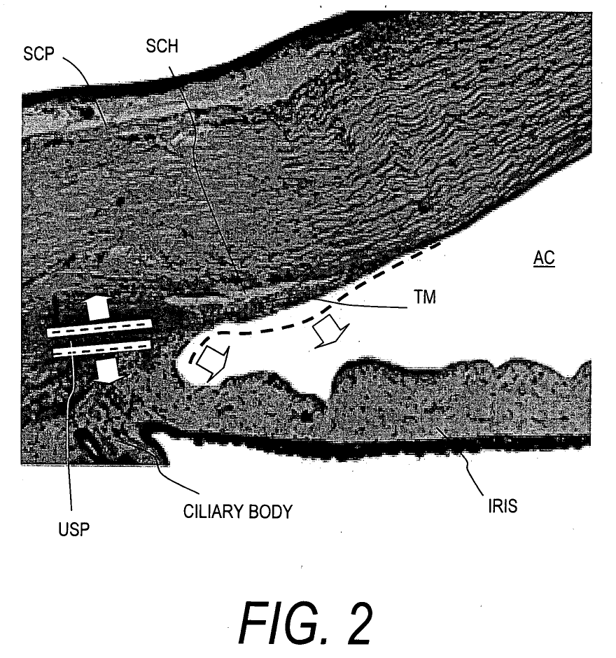

1. Type “A” implant for modifying tissue properties, geometry and permeability. FIG. 1 illustrates the anterior segment of a patient's eye wherein aqueous humor exits the anterior chamber AC in outflow pathways through the trabecular meshwork TM overlying Schlemm's canal SCH and through the uveoscleral plane USP. In general, the apparatus and method of the invention relate to stents or implants for the “retraction” of tissue in or about the aqueous outflow pathways proximate to the angle of the anterior chamber AC to enhance fluid outflows. By the term “retraction”, it is meant herein that the implant applies retracting forces to cause a change in geometry in targeted tissues that will enhance outflows therethrough by increasing porosity, expanding open spaces, increasing fluid permeability, expanding outflow channel cross-sections and the like. As will be described below, the stent has dual functionality in that it applies forces to cause such tissue retraction, and also can be configured with interior flow pathways through porosities, channels and the like to further facilitate fluid flow through the targeted tissue volumes. FIG. 2 is a sectional view of the angle of an anterior chamber AC generally showing the meshwork TM, Schlemm's canal SCH and uveoscleral plane USP in the tissue region that transitions into the angle. As indicated in FIG. 2, the tissues about the outflow pathways are targeted for retraction in the direction of the arrows, wherein the somewhat impermeable layers of the trabecular meshwork TM and posterior wall of Schlemm's canal SCH can be retracted inwardly within the anterior chamber. A stent also can be introduced into tissues in the uveoscleral plane USP to cause slight expansion thereof in the region of a stent body.

In general, several exemplary stents 100A-100G corresponding to the invention comprise a body with an extended or expanded “memory” shape that can be maintained in a “temporary” compacted or non-extended shape for introduction into the targeted tissue volume. Thereafter, energy that is stored within the constrained stent is released in the temporary-to-memory shape change to thereby apply retraction forces to the tissue. The terms “extended” and “non-extended” shapes are used herein for convenience when describing the shapes of an exemplary stent. The term “extended” is interchangeable with, and can have the same meaning as the terms “expanded”, “deployed”, “lengthened” or “elongated” etc. depending on the particular stent embodiment. The term “non-extended” as used herein is interchangeable herein with the terms “compacted”, “constrained”, “compressed”, “constricted” or “non-deployed”.

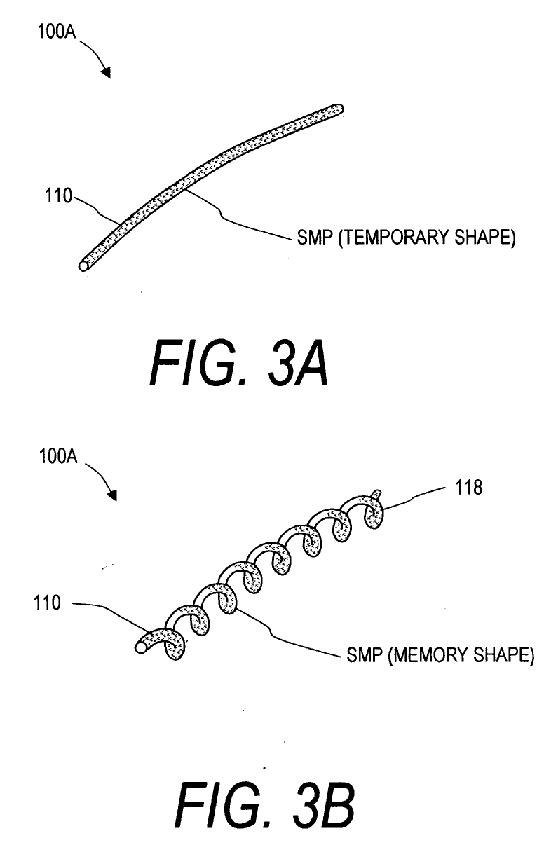

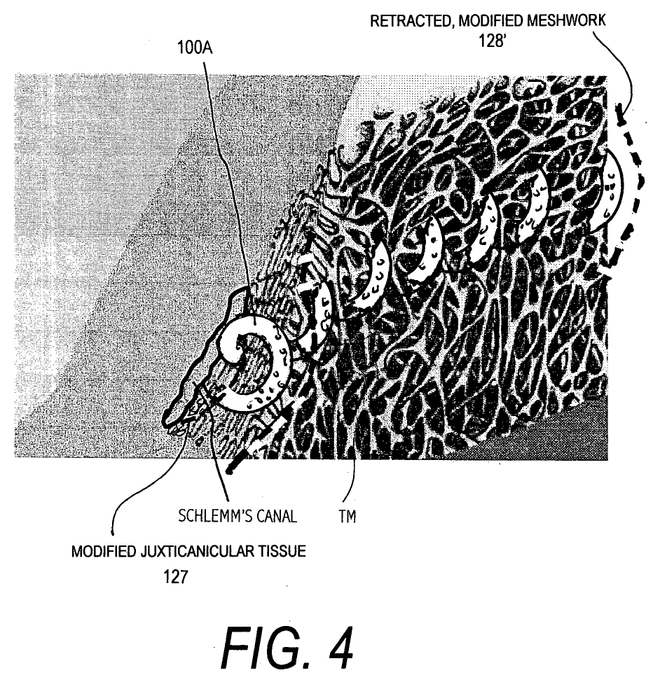

One preferred embodiment of an exemplary stent or implant 100A is illustrated in FIGS. 3A, 3B and 4 wherein the stent body 110 is of a shape memory polymer (SMP) extending along axis 115. The stent 100A has a first temporary reduced cross-sectional shape (FIG. 3A) for introduction into the outflow pathway tissue and a second expanded cross-sectional shape (FIG. 3B) for retracting or expanding the targeted tissue (see FIG. 4). In this embodiment, the memory shape is a spiral form as depicted in FIG. 3B. The stent body 110 can be a fluid impermeable polymer, or more preferably the SMP body has pores 122 therein for allowing fluid flow though the body. By this means, the stent 100A “retracts” the meshwork geometry to allow fluid flows therethrough in newly expanded spaces within the trabecular cords and sheets, while at the same time the interior of the stent body itself provides flow passageways.

FIG. 4 illustrates stent 100A after initial implantation in the eye and after expansion of the stent to its extended shape to retract tissue. The stent 100A further has an introducer device 124 (phantom view in FIG. 1) for assisting in the axial or helical introduction of the stent into a targeted location. As indicated in FIG. 1, the introduction of the stent can be in any suitable direction or angle through the cornea or sclera (e.g., introducer paths 125, 125′ and 125″). In some embodiments, as will be described below, the tissue modifying stents can be coupled to an extension portion for extending the stent's interior outflow path outwardly through the sclera, for example to the subconjunctival plane SCP. In one such embodiment, the outflow end of the extension portion will terminate within a region of the subconjunctival plane SCP generally radially inward of the lymphatic vessel network indicated at 105 in FIG. 1. In co-pending U.S. patent application Ser. No. 10/759,797 filed Jan. 17, 2004 (Docket No. S-IOS-00200) titled Implants for Treating Ocular Hypertension, Methods of Use and Methods of Fabrication, the author discloses various stents for providing outflows into the subconjunctival lymphatic network 105, which comprises three regions. First, there are localized “nets” or networks of small lymphatic vessels indicated at LN in FIG. 1. Second, there are lymphatic circumferentials LC (FIG. 1) that extend 180° or more generally parallel to the limbus that drain the localized lymphatic nets LN. Third, there are two large lymphatic vessels LV (FIG. 1) at about 6 and 12 o'clock that drain the circumferential vessels LC and extend posteriorly within the scleral surface to drain the lymphatic network. These circumferential vessels extend deep into the orbit of the eye. Daljit Singh, M. D., has investigated and described the lymphatic system of the eye (see, e.g., as of Sep. 27, 2003: webpage at http://www.ophmanagement.com/pf_article.asp?article=85458).

The exemplary tissue-modifying stent 100A can be fabricated of a substantially fluid impermeable biocompatible SMP polymer that can be deformed to an elongated linear shape as in FIG. 3A. Of particular interest, the stent 100A also can be fabricated of a shape memory polymer that is porous or is a SMP foam, which is sometimes called a CHEM. In any of these SMP embodiments, the implant can be deformed or compacted to a temporary non-extended shape and advanced into position in its temporary reduced cross-sectional configuration. For example, the stent can be compacted into the form of a cylinder, rod or low profile helically threaded body for implantation, with or with out a sharp tip or barbed tip. The stent 100A of FIG. 3A can be introduced from the bore of a needle introducer, or the stent can comprise a sleeve over a needle introducer. The stent can be pushed from the needle with a pusher member or the distal end can carry a barb element.

The stent 100A of FIG. 3A can be any dimension suitable for utilizing its outer surfaces to engage and retract tissue to expand spaces in the meshwork TM and Schlemm's canal SCH or in the region of the uveoscleral plane USP. A plurality of stents 100A can be introduced from a single introducer. The scope of the invention includes any diameter body from the 0.2 to 500 micron range. In another embodiment, the stent body 110 can have microfabricated interior flow channels and ports in the body that communicate with flow channels. In another embodiment and method of the invention, the stent body 110 is of a biodegradable or resorbable polymer that can be used for a temporary period of time to evaluate changes in outflow. By this means, non-resorbable or less resorbable versions can be implanted that provide different geometries for tissue retraction. The stent 100A also can be compacted into the form of a rod or other shape described above for deployment from a needle with an ultrathin constraining sleeve (e.g., a perforated sleeve) of a biodegradable polymeric material. Thus, thermal or biodegradable means can be used to releaseably maintain the stent 100A in the reduced cross-sectional shape. FIG. 4 illustrates the method of causing the stent to modify the properties of, and retract, the juxticanicular tissue 127. This tissue is the dense, compacted mesh layers adjacent Schlemm's canal SCH and expand or retract the meshwork into the anterior chamber indicated by initial profile 128 and modified meshwork profile 128′.

In order to better describe stent 100A that is fabricated of a SMP, it is first useful to provide a background on shape memory polymers. SMPs demonstrate the phenomena of shape memory based on fabricating a segregated linear block co-polymer, typically of a hard segment and a soft segment. The shape memory polymer generally is characterized as defining phases that result from glass transition temperatures (T_{g}) in the hard and soft segments. The hard segment of a SMP typically is crystalline with a defined melting point, and the soft segment is typically amorphous, with another defmed transition temperature. In some embodiments, these characteristics may be reversed together with the segment's glass transition temperatures. Some SMPs that are suitable for the implant are a subset of shape memory polymer material that comprises a foam polymer. In one known use of such a foam SMP, the material has been more particularly described as a cold-hibernated elastic memory (CHEM) polymeric foam that can be compacted into a temporary shape.

Referring to FIGS. 3A and 3B, the stent can be fabricated to provide a memory shape, such as 3-D shape of stent 100A in FIG. 3B. In such an embodiment, when the SMP material is elevated in temperature above the melting point or glass transition temperature of the hard segment, the material is then formed into its memory shape. The selected shape is memorized by cooling the SMP below the melting point or glass transition temperature of the hard segment. When the shaped SMP is cooled below the melting point or glass transition temperature of the soft segment while the shape is deformed, that temporary shape is fixed as in FIG. 3A. The temporary shape can be a highly compacted shape for introduction (such as a cylinder or helical form with shallow threads for axially or helically inserting into tissue) and maintained in the compacted state without a constraining sleeve member.

The original memory shape is recovered by heating the material above the melting point or glass transition temperature T_{g} of the soft segment but below the melting point or glass transition temperature of the hard segment. (Other methods for setting temporary and memory shapes are known which are described in the literature below). The recovery of the original memory shape is thus induced by an increase in temperature, and is termed the thermal shape memory effect of the polymer. The transition temperature can be body temperature or somewhat below 37° C. for a typical embodiment. Alternatively, the SMP can be designed to have a higher transition temperature and a remote energy source can be used to elevate the temperature and expand the SMP structure to its memory shape (e.g., inductive heating or light energy absorption). Thus, the scope of the invention includes utilizing light energy or inductive heating to cause the temporary-to-memory transition of the stent 100A.

Besides utilizing the thermal shape memory effect of the polymer, other memorized physical properties of the SMP can be controlled by its change in temperature or stress, particularly in ranges of the melting point or glass transition temperature of the soft segment of the polymer, e.g., the elastic modulus, hardness, flexibility, permeability and index of refraction. Examples of polymers that have been utilized in hard and soft segments of SMPs include polyurethanes, polynorborenes, styrene-butadiene co-polymers, cross-linked polyethylenes, cross-linked polycyclooctenes, polyethers, polyacrylates, polyamides, polysiloxanes, polyether amides, polyether esters, and urethane-butadiene co-polymers and others identified in the following patents and publications: U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al. (all of which are incorporated herein by reference); Mather, Strain Recovery in POSS Hybrid Thermoplastics, Polymer 2000, 41(1), 528; Mather et al., Shape Memory and Nanostructure in Poly(Norbonyl-POSS) Copolymers, Polym. Int. 49, 453-57 (2000); Lui et al., Thermomechanical Characterization of a Tailored Series of Shape Memory Polymers, J. App. Med. Plastics, Fall 2002; Gorden, Applications of Shape Memory Polyurethanes, Proceedings of the First International Conference on Shape Memory and Superelastic Technologies, SMST International Committee, pp. 120-19 (1994); Kim, et al., Polyurethanes having shape memory effect, Polymer 37(26):5781-93 (1996); Li et al., Crystallinity and morphology of segmented polyurethanes with different soft-segment length, J. Applied Polymer 62:631-38 (1996); Takahashi et al., Structure and properties of shape-memory polyurethane block copolymers, J. Applied Polymer Science 60:1061-69 (1996); Tobushi H., et al., Thermomechanical properties of shape memory polymers of polyurethane series and their applications, J. Physique IV (Colloque C1) 6:377-84 (1996)) (all of the cited literature incorporated herein by this reference). The above background materials, in general, describe SMP in a non-open cell solid form. The similar set of polymers can be foamed, or can be microfabricated with an open cell structure for use in the invention. See Watt A. M., et al., Thermomechanical Properties of a Shape Memory Polymer Foam, available from Jet Propulsion Laboratories, 4800 Oak Grove Drive, Pasadena Calif. 91109 (incorporated herein by reference).

Shape memory polymers foams that fall within the scope of the invention typically are polyurethane-based thermoplastics that can be engineered with a wide range of glass transition temperatures. These SMP foams possess several potential advantages for intraocular implants, for example: very large shape recovery strains are achievable, e.g., a substantially large reversible reduction of the Young's Modulus in the material's rubbery state; the material's ability to undergo reversible inelastic strains of greater than 20%, and preferably greater than 50% with shape recovery at a selected temperature between about 30° C. and 60° C. This type of SMP also will allow for injection molding thus allowing stents with complex shapes. These polymers also demonstrate unique properties in terms of capacity to alter the material's water or fluid permeability and thermal expansivity. Thus, one stent of the invention consists of an SMP implant that changes its dimension and fluid permeability in response to temperature. However, the material's reversible inelastic strain capabilities leads to its most important property—the shape memory effect. If the polymer is strained into a new shape at a high temperature (above the glass transition temperature T_{g}) and then cooled it becomes fixed into the new temporary shape. The initial memory shape can be recovered by reheating the foam above its T_{g}.

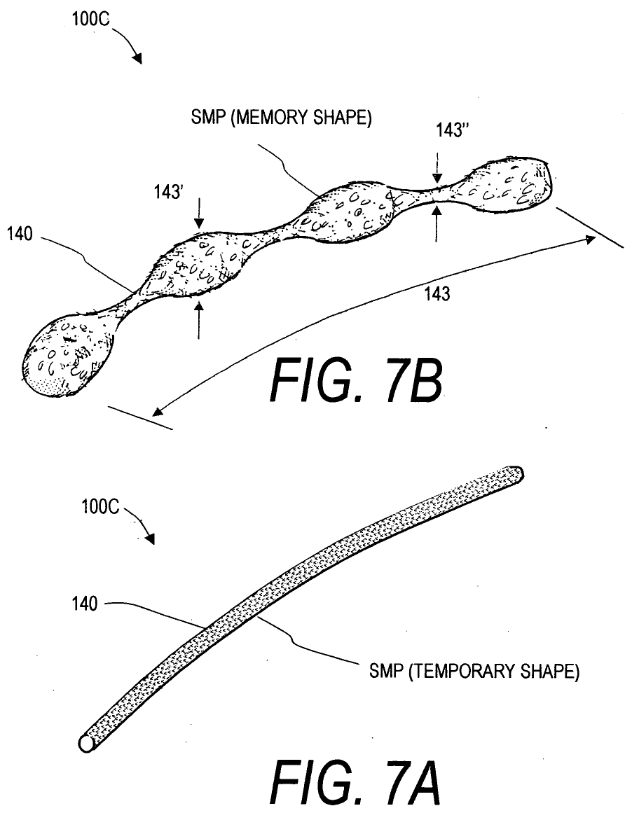

In any embodiment of polymer stent (e.g., FIGS. 3A and 3B) as described above, the polymeric body 110 can be micro- or nano-fabricated using soft lithography techniques to provide an open or channeled interior structure to allow fluid flow therethrough. The interior channels can be molded in layers assembled by soft lithographic techniques. Stents can be micro-fabricated of a resilient polymer (e.g., silicone) by several different techniques collectively known as soft lithography. For example, microtransfer molding is used wherein a transparent, elastomeric polydimethylsiloxane (PDMS) stamp has patterned relief on its surface to generate features in the polymer. The PDMS stamp is filled with a prepolymer or ceramic precursor and placed on a substrate. The material is cured and the stamp is removed. The technique generates features as small as 250 nm and is able to generate multi layer systems that can be used to fabricate the stent as well as flow channels therein. Replica molding is a similar process wherein a PDMS stamp is cast against a conventionally patterned master. A polyurethane or other polymer is then molded against the secondary PDMS master. In this way, multiple copies can be made without damaging the original master. The technique can replicate features as small as 30 nm. Another process is known as micromolding in capillaries (MIMIC) wherein continuous channels are formed when a PDMS stamp is brought into conformal contact with a solid substrate. Then, capillary action fills the channels with a polymer precursor. The polymer is cured and the stamp is removed. MIMIC can generate features down to 1 μm in size. Solvent-assisted microcontact molding (SAMIM) is also known wherein a small amount of solvent is spread on a patterned PDMS stamp and the stamp is placed on a polymer, such as photoresist. The solvent swells the polymer and causes it to expand to fill the surface relief of the stamp. Features as small as 60 nm have been produced. A polymeric microstructure as in a stent can be entirely of a “Lincoln-log” type of assembly similar to that shown in Xia and Whitesides, Annu. Rev. Mater. Sci. 1998 28:153-84 at p. 170 FIG. 7d (the Xia and Whitesides article incorporated herein by reference).

In any embodiment of SMP stent, the polymer can have a “surface modification” to enhance fluid flows therethrough, and to prevent adherence of body materials to the surfaces of the outflow pathway. Such surface modifications are known in the art, for example as provided in technologies developed by SurModics, Inc., 9924 West 74th St., Minneapolis, Minn. In another surface modification, phosphotidylcholine based coatings have been developed to prevent protein adhesion and are known in the art.

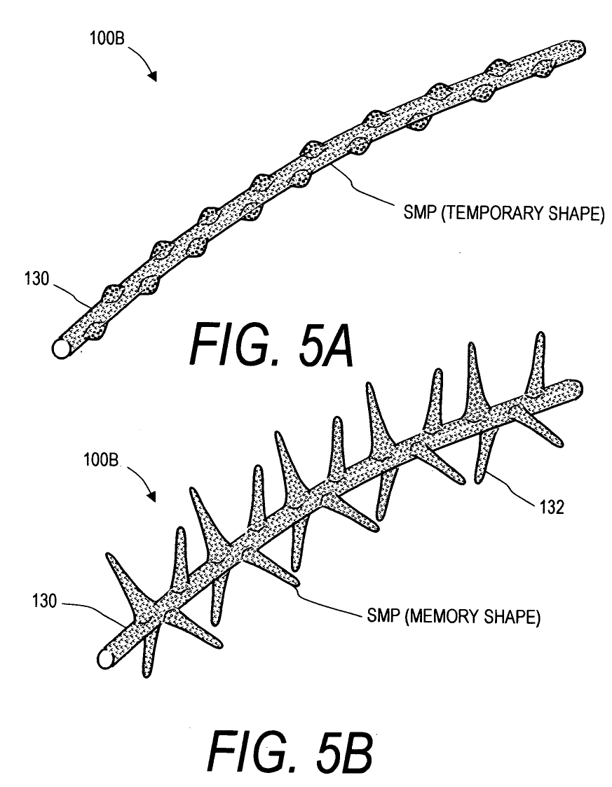

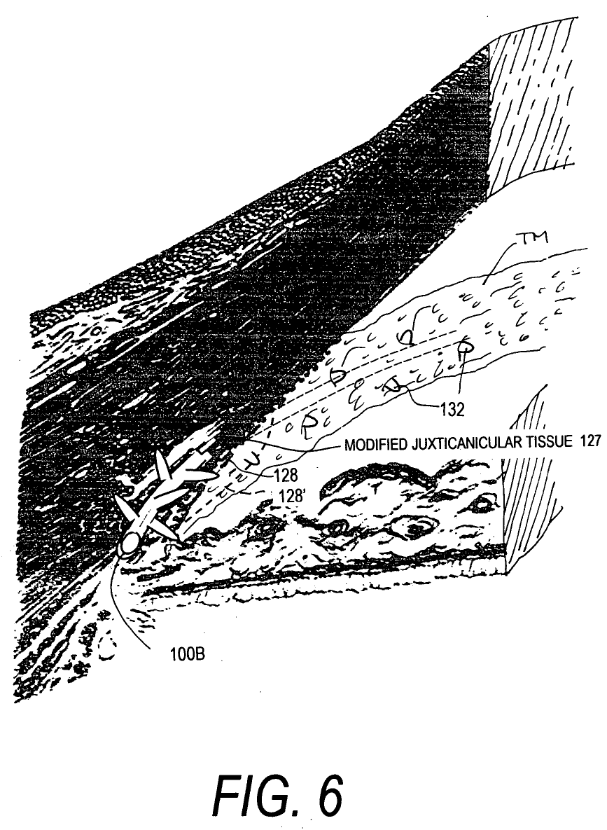

FIGS. 5A, 5B and 6 illustrate another preferred embodiment of SMP stent 100B that functions similarly to the stent of FIGS. 3B and 4 to retract and modify tissue. More in particular, the stent retracts tissue in the trabecular meshwork to expand open spaces and flow pathways therein. The polymer stent 100B also can be fabracated by soft lithography techniques with an interior flow channel structure by means described above. In one embodiment, the axial body portion 130 has a plurality of projecting elements or spikes 132 in the memory shape of FIG. 5B. The spikes 132 can be compacted to provide a memory shape similar to that of FIG. 5A. FIG. 6 illustrates the stent 100B in the trabecular meshwork tissue. The spikes can be of any number and oriented rnadomly so that some will extend toward the anterior chamber AC and some will extend into, or toward, Schlemm's canal SCH. The stent spikes 132 can expand spaces within the meshwork as well as carry interior flow channels or additional surface relief 135 to provide surface flow channels. The spikes 132 and body portion 130 can be of any suitable dimension for the introduction of one or more such stents 100B into the meshwork with a needle-type introducer (cf. FIG. 1). In the method of the invention, the stent can advantageously be deployed in a path generally in the meshwork and can retract tissue no matter whether portions of the stent are in the meshwork, Schlemm's canal or both. In FIG. 4, its can be seen that the juxticanicular tissue 127 and the meshwork surface 128′ are modified and retracted.

In another embodiment, the spikes 132 can be of different SMP compositions and/or dimensions to store differential amounts of energy to thereby release greater and lesser energy when moving toward the memory shape (FIG. 5B) . The stent can be oriented in its introduction to cause particular spikes 132 that apply greater expansion forces to be oriented toward Schlemm's canal. The spikes 132 that apply lesser expansion forces can be oriented to expand toward the anterior chamber AC.

In any embodiment of SMP, the stent body can have different portions fabricated of different SMP compositions to provide differential time intervals for energy release. Thus, the stent can provide delayed timing of the application of retraction forces. The post-time implant time-control of retraction forces is provided according to the invention in three manners. First, a preferred method is to provide selected features or portions of the stent (e.g., spikes 132 in FIGS. 5A-5B) of SMP portions that require different stimuli or different stimulus parameters to release stored energy in the different features. For example, the some spikes 132 in FIGS. 5A-5B are fabricated of a shape memory polymer that moves to the extended shape at body temperature. Other spikes 132 as in FIGS. 5A-5B are of a SMP that moves to the extended shape at above body temperature, for example in the 40°-46° C. range and are actuated by a light source or laser that cooperates with a selected chromophore in the spike. A plurality of different spikes 132 can be provided that respond to different wavelengths, again by fabricating the spikes with different cooperating chromophores. Additionally, the SMP in any feature or region can be designed to provide of continuum of shape changes depending on a parameter of the stimulus that is used to actuate the SMP. In one example, light energy of differing time intervals or power levels can be used to move an SMP feature to a selected shape between its temporary shape and its memory shape. In another embodiment, the SMP feature can be elevated in temperature by providing ferromagnetic or another magnetic responsive particle therein that responds to inductive heating from an external source as is known in the art.

In a second manner of providing post-implant timed application of retracting forces, the stent is provided with different SMP portions that expand at different rates, for example some stent portions expand immediately at body temperature and some portions expand more slowly to the memory shape at body temperature. In a third manner of providing post-implant timed application of retracting forces, the extendable portions of the stent can have surface layers or constrianing portions of a different bioerodible polymers, with each extendable portion thus being programmed to extend at a different time post-implant.

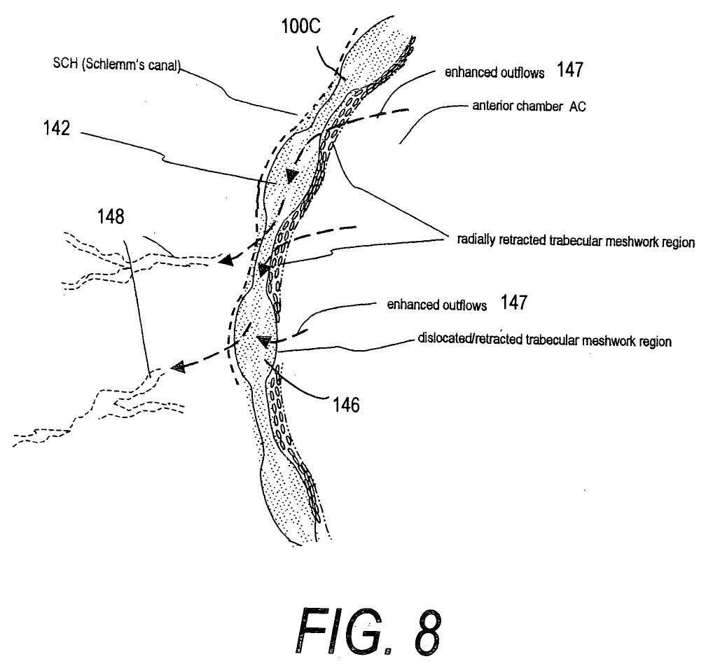

FIGS. 7A, 7B and 8 illustrate another embodiment of SMP stent 100C that retracts the trabeculum in a somewhat different manner than stents 100A and 100B described above. The stent body 140 can be of porous shape memory polymer foam as described above and have a memory shape with a series of undulations 142 (FIG. 7B). The axial and cross-sectional dimensions 143, 143′ and 143″ can be any dimension suitable for deploying the stent in the meshwork. Alternatively, the stent can be microfabricated by soft lithography means with interior flow channels. The stent can be introduced into or adjacent to Schlemm's canal SCH or generally within the meshwork TM. As the stent moves to its extended position as indicated in FIG. 8, the retraction forces will dislocate and retract the meshwork to thereby modify its permeability. The radially retracted regions 144 will then have increased open spaces 145. Further, portions of the porous stent surface indicated at 146 may be directly exposed to the anterior chamber AC. The enhanced outflows 147 are indicated by arrows in FIG. 8 wherein flows into Schlemm's canal SCH then communicate with collectors 148.

In an alternative embodiment, different axial portions of the stent 100C can be of different SMPs to provide the differential post-implant timing of the retraction forces to optimize outflow paths differently over time. In this type of stent 100C, the use of a laser for “actuating” a different axial SMP portion to its extended shape on demand would be useful. As described above, stent 100C like the other embodiments also can be of a resorbable material that is designed to degrade and disappear over a selected time interval.

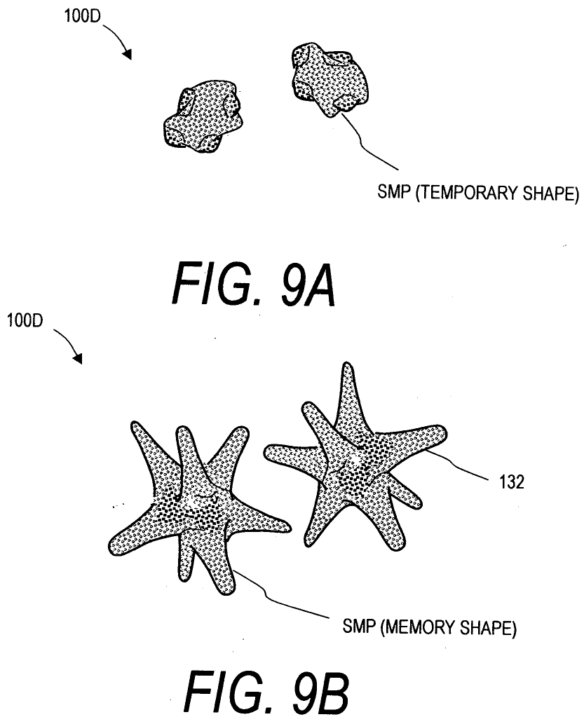

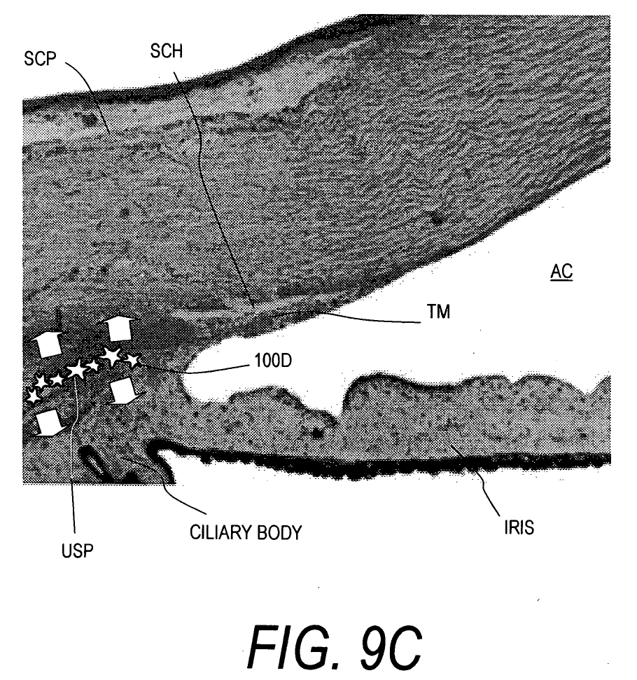

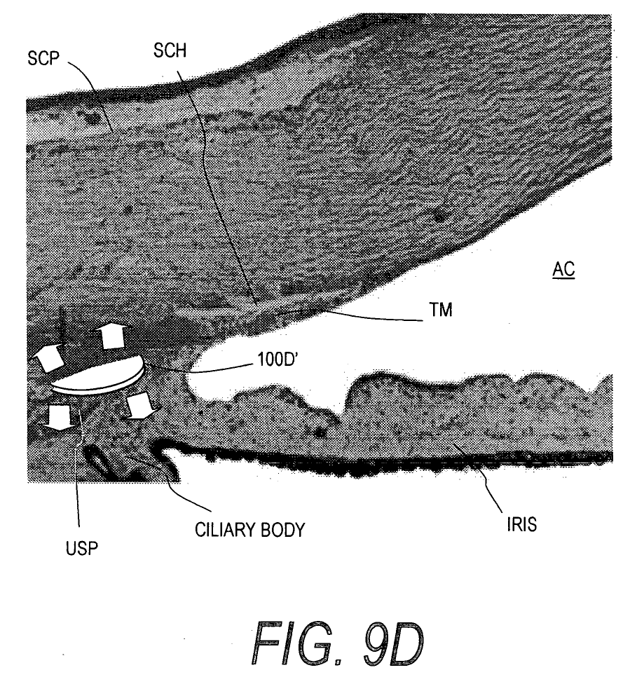

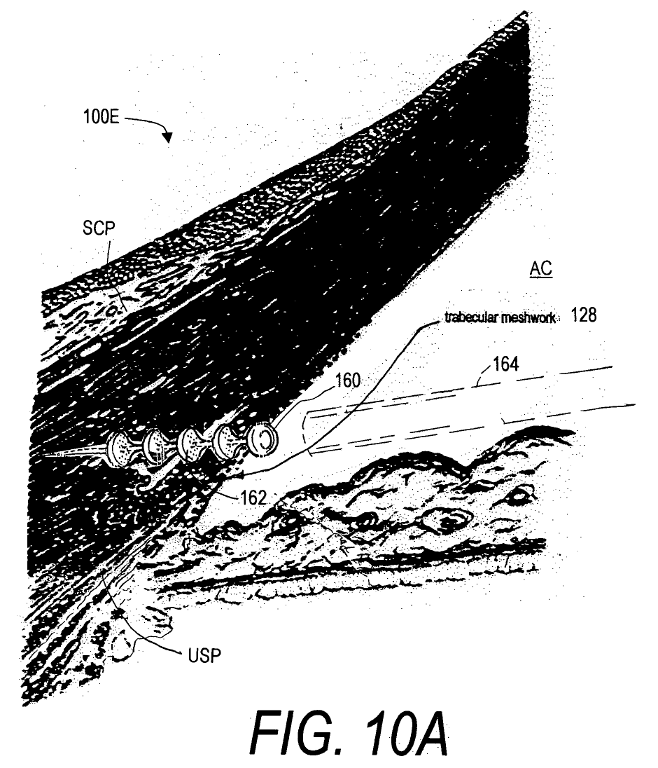

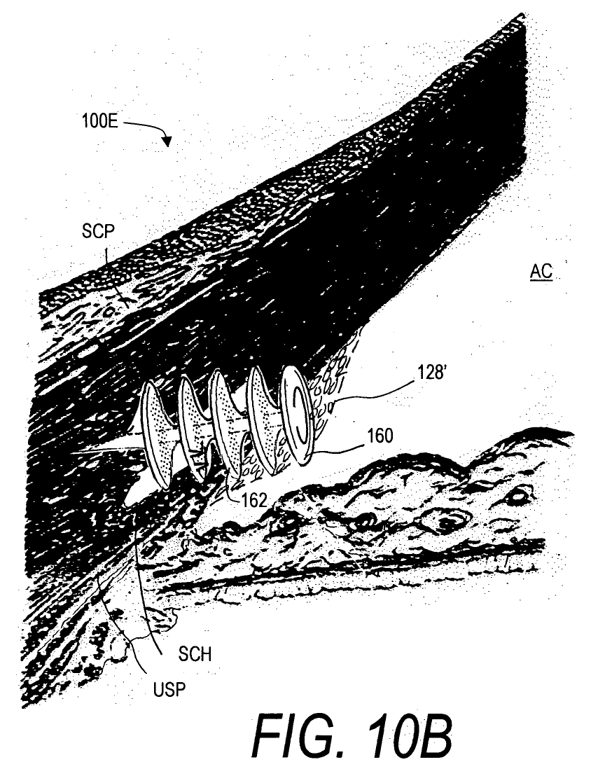

FIGS. 9A and 9B illustrate another embodiment of SMP stent 100D that functions as distributed tissue retracting system and soft tissue dissecting system wherein a volume or plurality of implantable SMP bodies 150 are provided for introduction into the meshwork or the uveoscleral plane USP. In use, the expansion of the plurality of bodies will self-locate and release stored energy in the transition from the temporary shape (FIG. 9A) to the memory shape (FIG. 10B) to retract tissues and preferentially dissect a tissue plane along the plane of least resistance to provide fluid permeable outflow pathways in tissue. The cross-sections of the SMP bodies 150 can be any suitable dimension from 100 nm upwards to 1000 microns or more. The spikes 132 are compacted to provide a memory shape as in FIG. 9A. In one method of use, the stent system 100D and can be introduced into the meshwork region wherein the spikes 132 will expand through the trabecular sheets which is in all respects similar to the embodiment depicted in FIG. 6. FIG. 9C depicts a preferred method of use wherein a volume of implants 100D (not-to-scale) are introduced into the region of the uveoscieral plane USP wherein expansion of the SMP bodies 100D causes distributed dissection of the uveoscleral plane which will be a plane of least resistance thereby increase its fluid permeability. The stent bodies 100D are preferably of an open cell SMP foam that allow fluid migration therethrough bodies. The stent bodies 100D further are preferably of a bioabsorbable polymer that degrades over time. The plurality of bodies 100D can further expand and degrade at varied rates to provide a time-controlled and extended period of actuation and bioabsroption to thereby allow repeated treatments over an extended period of time. In another embodiment for retracting the uveoscleral plane as in FIG. 9D, the SMP stent 100D′ can have any suitable planar form in its memory shape to dissect and retract the uveoscleral plane, such as a round disc shape. The stent can have interior flow passageways as described above. Such a stent can have any deformed temporary shape such as an elongated cylinder.

The scope of the invention extends to introduction of SMP bodies 150 as in FIGS. 9A-9B into any targeted soft tissue volume or plane in the body to thereafter release the stored energy to apply retracting forces and thereby modify one or more tissue properties. Again, this embodiment of the invention lends itself to providing different bodies with different SMPs for post-implant timed release of retracting forces, whether by external means (e.g., laser actuation) or internal means (biodegradable constraint portion). The method of the invention includes using the SMP body or bodies to modify a tissue property such as tissue permeability (i.e., intra-tissue fluid flow properties), tissue density, tissue resilience, tissue orientation, bulk (or mass) and flexibility of the soft tissues. The use of SMP stents or SMP bodies in general for retracting tissue, retracting and treating body cavities, dissecting tissue planes and delivering energy to tissue planes and body cavities was first disclosed in the author's patent application Ser. No. 60/422,646 filed Oct. 31, 2002 titled Electrosurgical System Utilizing Thermoscissile Polymeric Compositions, which is incorporated herein by this reference.

FIGS. 10A and 10B illustrate another SMP stent 100E that retracts the trabeculum in with a stent body 160 that can be introduced in a radial direction across the meshwork TM. The stent body 160 again can be of a porous shape memory polymer foam (or CHEM) and have a memory shape formed into a series of threads, flanges or undulations 162 (FIG. 10B). The stent also can be microfabricated by soft lithographic means with interior flow channels. In FIG. 10A, the stent body is deployed from a needle-like introducer 164 and is shown in a slightly expanded state as it expands away from its temporary non-extended shape. As can be seen in FIG. 10B, the stent body 160 expands to its memory shape thereby applying retracting forces to radially retract layers of trabecular cords toward the anterior chamber AC from initial profile 128 to modified meshwork profile 128′, thereby expanding outflow pathways.

In any embodiment of SMP stent 100A-100E above, the surface of the stent can carry surface relief, asperities, grooves and the like for causing fluid flows along and about the stent surfaces. Any stent also can be provided with hydrophobic or hydrophilic surfaces for causing interactions with body fluid to ehance fluid outflows.

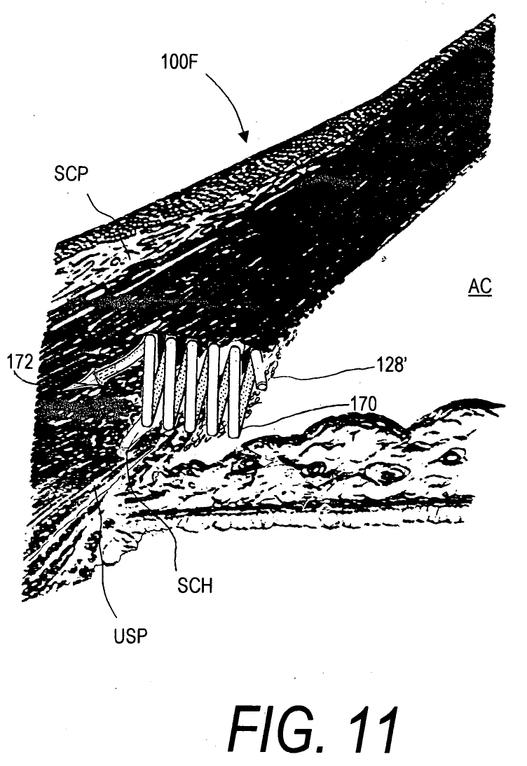

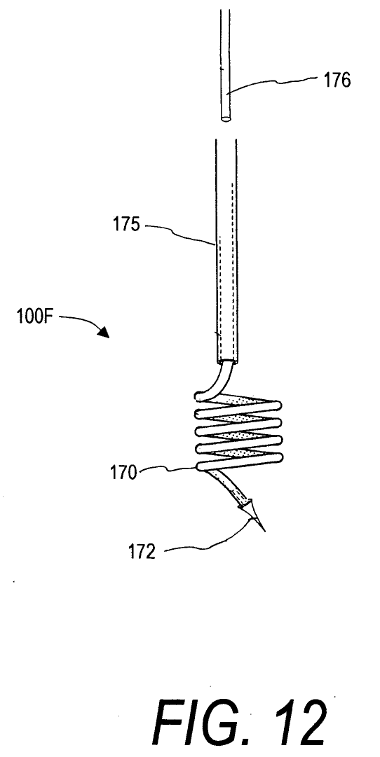

Referring now to FIGS. 11 and 12, an alternative stent 100F has a body 170 that again is of a “memory” material for using stored energy to retract tissue. The stent 100F differs in that it is fabricated of a shape memory alloy (SMA) in a helical form about its axis. Optionally, there is a lumen in the stent body 170. The stent's length and diameter are suitable for placement in the trabecular meshwork TM and Schlemm's canal SCH. In one embodiment, the tip 172 or distal portion is of a bioresorbable polymer. FIG. 11 illustrates the stent body 170 in its memory shape after being deployed in tissue which is similar to the SMP embodiment of FIG. 10B. The stent is maintained in a temporary shape in a bore of an introducer 175 as can be seen in FIG. 12. The barb-type tip 172 of the stent and/or the pusher member 176 is used to deploy the implant from the introducer-needle 175 as the introducer is moved retrograde. It can be easily understood how the implant can be introduced atraumatically into the meshwork TM and Schlemm's canal SCH as in FIG. 11. In FIG. 11, the retraction method of the invention is evident as the shape recovery of the SMA stent thereby causes its surfaces to gently engage the cords and sheets of the meshwork TM to thereby retract it to profile 128′ (FIG. 11) and increase the open region within the meshwork to enhance aqueous outflows.

In another stent embodiment of a shape memory alloy (SMA), the stent can have a body (not shown) with an undulating form (memory shape) similar to that of FIGS. 7A and 7B. The stent is fabricated of woven SMA filaments in a Chinese “finger-toy” configuration in its memory shape. The stent body can be introduced into the trabecular meshwork to retract and space apart the trabecular cords and sheets to enhance outflows to Schlemm's canal as in FIG. 8.

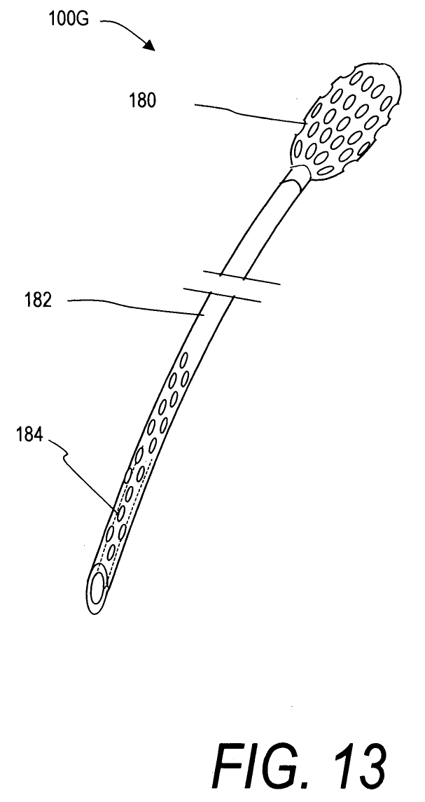

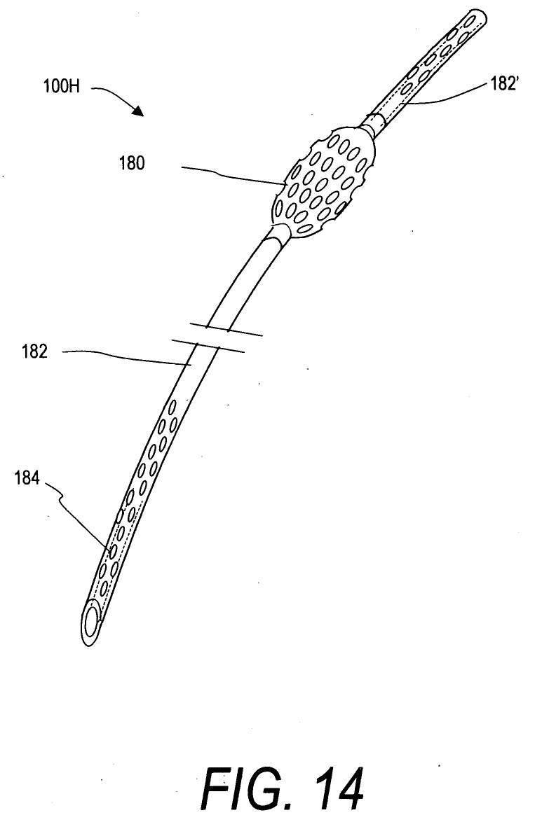

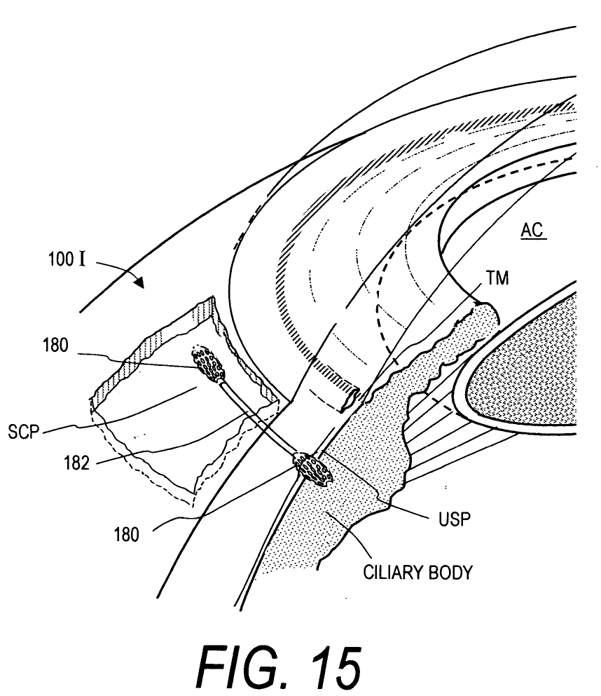

FIG. 13 illustrates an alternative stent body 100G with a first end comprising SMP portion 180 wherein the shape memory polymer is an expandable polymer foam that functions as described above to retract and modify tissue. The second end or extension portion 182 of the stent is of a polymer that has flow passageways 184 therein in the form of a lumen or an open cell material. In another embodiment, stent body 100H is illustrated in FIG. 14 wherein the SMP portion 180 is in a medial portion of the stent body with extension portions 182 and 182′ extending in opposing directions from the expandable SMP portion. In another embodiment (FIG. 15), the stent body 100I can have two SMP portions 180 at opposing ends of the stent with an extension member 182 therebetween. FIG. 15 depicts a stent 100I with expandable SMP portions in the uveoscleral plane USP and the subconjunctival plane SCP to provide distributed outflow proximate to the lymphatic network (see FIG. 1). The author disclosed similar embodiments in co-pending U.S. patent application Ser. No. 10/759,797 filed Jan. 17, 2004 titled Implants for Treating Ocular Hypertension, Methods of Use and Methods of Fabrication.

In any of the stents 100A-100I that carry at least one expandable SMP portion 180 coupled to an extension portion for carrying fluid flows, the inflow end of the stent is preferably implanted to retract and modify tissue in the region of the meshwork TM or uveoscleral plane USP although the inflow end can also extend into the anterior chamber. The outflow end preferably is implanted to retract and modify tissue in the region of the lymphatic net (see FIG. 1) in the subconjunctival plane SCP. Alternatively, the outflow end can be in the uveoscleral plane USP when the inflow end is within the meshwork region.

In any stent 100A-100I above, the stent body also can carry a drug-release surface coating as is known in the art. Agent eluting polymers have been developed by many companies, for example SurModics, Inc., 9924 West 74th St., Minneapolis, Minn. Any pharmacological agent classes that have been developed for treating glaucoma can be a candidate for release from the stent, for example latanoprost and other prostaglandins, timolol and other β-blockers, carbonic anhydrase inhibitors, miotics and sympathomimetics.

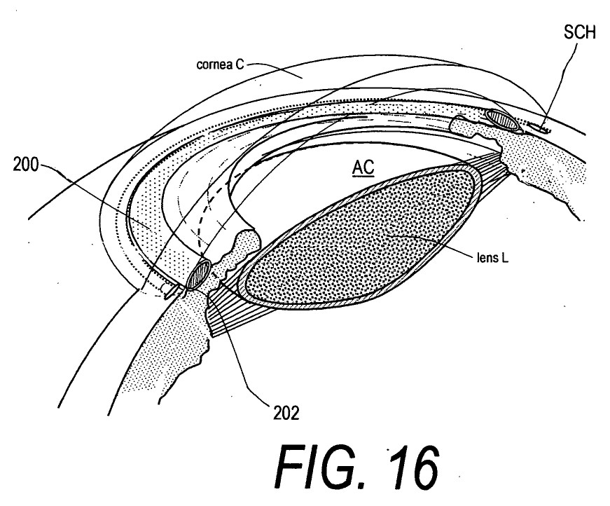

FIG. 16 illustrates another embodiment of a shape memory stent 200 of a different form for treating closed angle glaucoma. In one embodiment, the stent body 200 is a transparent SMP foam structure that extends entirely around the circumference of the anterior chamber AC to retract, expand and open the angle 202. In another embodiment, the body 200 is a transparent SMP polymeric open lattice structure that is microfabricated by soft lithography means as described above to extend around the circumference of the anterior chamber to retract, expand and open the angle. The stent can be compacted, folded and rolled for ease of introduction into the anterior chamber AC.

Those skilled in the art will appreciate that the exemplary embodiments and descriptions thereof are merely illustrative of the invention as a whole. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention.

What is claimed is:

1. A method for enhancing aqueous humor flows outwardly from an anterior chamber of a human eye to decrease intraocular pressure (IOP), the method comprising modifying a property of a targeted soft tissue volume in and about aqueous outflow pathways to increase fluid flow rates therethrough.

2. A method for enhancing aqueous humor flows as in claim 1 wherein the expanding step includes implanting at least one implant body in soft said tissue volume in a non-extended shape and extending the implant to an extended shape.

3. A method for enhancing aqueous humor flows as in claim 2 wherein the implanting step includes providing the at least one implant body of a shape memory material wherein its memory shape is the extended shape.

4. A method for enhancing aqueous humor flows as in claim 2 wherein the implanting step includes providing the at least one implant body of a shape memory polymer (SMP) having a memory extended shape and a temporary non-extended shape.

5. A method for enhancing aqueous humor flows as in claim 2 wherein the implanting step includes providing the at least one implant body of a polymer having a memory extended shape and a non-extended shape being constrained by a surface constraining portion.

6. A method for enhancing aqueous humor flows as in claim 2 wherein the extending step includes allowing a thermal stimulus to cause the at least one implant body to move to said extended shape.

7. A method for enhancing aqueous humor flows as in claim 6 wherein the thermal stimulus is provided by body heat.

8. A method for enhancing aqueous humor flows as in claim 6 wherein the thermal stimulus is provided by energy from an external source selected from the class consisting of light energy sources and inductive heating sources.

9. A stent for treating ocular hypertension comprising a stent body at least partly of a shape memory material capable of a temporary non-extended shape for introduction and a memory extended shape for applying retracting forces on tissue, the stent body dimensioned for implantation in the region of the aqueous outflow pathways proximate the angle of the anterior chamber.

10. A stent as in claim 9 wherein the shape memory material is a shape memory polymer (SMP).

11. A stent as in claim 9 wherein the shape memory material is a nickel titanium alloy.

12. A stent as in claim 9 wherein the shape memory material defines flow channels extending at least partly therethrough.

13. A stent as in claim 9 wherein the shape memory material is fluid permeable.

14. A stent as in claim 9 wherein the shape memory material is a SMP foam.

15. A stent as in claim 9 wherein the shape memory material is a soft lithography microfabricated body.

16. A stent as in claim 9 wherein the stent body carries surface relief elements.

17. A stent as in claim 9 wherein the stent body is at least partly bioabsorbable.

18. A stent as in claim 10 wherein the SMP has a polymer portion that defines a transition temperature at or below about 37° C. for permitting body temperature stimulus to its memory extended shape from its temporary non-extended shape.

19. A stent as in claim 10 wherein the SMP has a polymer portion that defines a transition temperature above about 37° C. for cooperating with an external source for moving to its memory extended shape from its temporary non-extended shape.

20. A stent as in claim 10 wherein the SMP carries a selected chromophore for cooperating with a light energy source for allowing non-invasive change in the temperature of the SMP.

21. A stent as in claim 10 wherein the SMP carries a magnetic responsive composition for cooperating with an external inductive source for allowing non-invasive change in the temperature of the SMP.

22. An implantable device for treating glaucoma in a human eye comprising a stent for retracting tissue about aqueous outflow channels of a human eye, the stent of at least one shape memory polymer composition.

23. An implantable device as in claim 22 wherein the stent defines a temporary non-extended shape and a memory extended shape for retracting said tissue.

24. An implantable device as in claim 22 wherein the stent is of a shape memory polymer composition is substantially transparent.

25. An implantable device as in claim 22 wherein the stent has a form in its memory extended shape has at least one of threads, projections, ridges, spikes, undulations, convolutions, grooves, surface relief and spiral portions.

26. A method for retracting soft tissue, the method comprising the steps of providing at least one SMP body capable of a first memory shape and a second temporary stressed shape that stores energy, implanting the at least one SMP body in the targeted tissue, and allowing a stimulus to move the SMP body to the first memory shape from the second temporary shape thereby releasing the stored energy to retract tissue.

27. A method as in claim 26 wherein the stimulus is body temperature.

28. A method as in claim 26 wherein the stimulus is energy from an external source selected from the class consisting of light energy sources and inductive heating sources.

29. A method for modifying a property of targeted soft tissue region in a human subject, comprising the steps of: providing a shape memory polymer (SMP) body in a shape that maintains therein reversible inelastic strains of greater than 20%; introducing the SMP body into the vicinity of said tissue region; and allowing a stimulus to release said inelastic strains in the SMP body to thereby apply forces that modify a property of said tissue region.

30. A method as in claim 30, wherein the property includes at least one of the properties in the class consisting of permeability, density, resilience, orientation, bulk and flexibility of said tissue region.

31. A stent for treating ocular hypertension in a human eye comprising a body of a shape memory polymer capable of reversible inelastic strains of greater than 20%.
